(19) United States
(12) Patent Application Publication
(10) Pub. No.: US 2026/0232555 A1
(43) Pub. Date: Aug. 13, 2026

(54) Personal Care Compositions

(71) Applicant: Colgate-Palmolive Company, New York, NY (US)

(72) Inventors: Yanjun DENG, Guangzhou (CN); Dongyao HUANG, Guangzhou (CN); Yuyan ZENG, Guangzhou (CN)

(73) Assignee: Colgate-Palmolive Company, New York, NY (US)

(21) Appl. No.: 19/160,541

(22) PCT Filed: Mar. 8, 2024
(86) PCT No.: PCT/US2024/019096

§ 371 Date: Aug. 28, 2025

(30) Foreign Application Priority Data
Mar. 8, 2023 (CN) 202310217264.1
Mar. 8, 2023 (CN) 202310217268.X

(51) Int. Cl. A61K 8/44; A61K 8/365; A61K 8/46; A61K 8/60; A61K 8/86; A61Q 19/10

(52) U.S. Cl. CPC A61K 8/442; A61K 8/365; A61K 8/466; A61K 8/602; A61K 8/86; A61Q 19/10; A61K 2800/30; A61K 2800/524

(57) ABSTRACT

Personal care compositions are disclosed herein. In accordance with an aspect of the disclosure, provided is a personal care composition having from about 1 to about 26 wt. % of a surfactant system comprising a non-sulfate anionic surfactant, an amphoteric surfactant, and a nonionic surfactant; and a polyol, wherein the personal care composition has a pH of about 2 to about 4.5, and all weight percentages are based on the total weight of the personal care composition.

### CROSS-REFERENCE TO RELATED APPLICATIONS

This application claims the benefit of priority from Chinese Patent Application No. 202310217264.1, filed Mar. 8, 2023, titled Personal Care Compositions, and from Chinese Patent Application No. 202310217268.X, filed Mar. 8, 2023, titled Personal Care Compositions, the contents from both of which are hereby incorporated herein in their entireties, for all purposes.

### BACKGROUND

Personal care composition products like body washes are becoming more popular in the United States and around the world. Desirable body wash compositions must meet a number of criteria. For example, in order to be acceptable to consumers, a body wash composition must exhibit good cleaning properties, must exhibit good lathering characteristics, must be mild to the skin (not cause drying or irritation) and preferably should even provide a conditioning benefit to the skin.

Body washes (sometimes referred to as shower gels) are a general term used to describe liquid surfactant containing formulations used to clean the body. While sometimes considered generically as “soap”, such body wash formulations frequently do not contain soaps such as sodium or potassium salts of fatty acids. Conventional body wash formulations, typically contain one of a variety of components including one or more surfactants, various emollients, fragrances and other personal care ingredients. Conventional body washes offer less skin irritation, improved lather in hard water conditions, and leave less residues on the skin and bathroom fixtures when compared with common soaps. Conventional body wash formulations use an anionic surfactant, typically sodium laurel sulfate or sodium laureth sulfate (SLES).

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

In accordance with an aspect, provided is a personal care composition having from about 1 to about 26 wt. % of a surfactant system comprising a non-sulfate anionic surfactant, an amphoteric surfactant, and a nonionic surfactant; and a polyol, wherein the personal care composition has a pH of about 2 to about 4.5, and all weight percentages are based on the total weight of the personal care composition.

The surfactant system of the personal care composition may be present in an amount from about 1 to about 20 wt. %. In at least one embodiment, the non-sulfate anionic surfactant is present in an amount from about 0.3 to about 10 wt. %. The amphoteric surfactant may be present in an amount from about 1 to about 9 wt. %. Additionally or alternatively, the nonionic surfactant may be present in an amount from about 1 to about 11 wt. %. The personal care composition may have a weight ratio of non-sulfate based anionic surfactant to amphoteric surfactant from about 1:2 to about 1:9. For instance, the personal care composition may have a weight ratio of the taurate surfactant to betaine surfactant from about 1:2 to about 1:9.

In certain embodiments, the surfactant system may comprise a non-sulfate anionic surfactant is selected from a sulfonate surfactant, a taurate surfactant, an isethionate surfactant, a sarcosinate surfactant, a sulfonate surfactant, a sulfosuccinate surfactant, a sulfoacetate surfactant, an alkoxylated monoacid surfactant, a salt thereof, and a combination of two or more thereof. The non-sulfate based anionic surfactant may comprise a taurate surfactant having an aliphatic carbon chain of 8 to 21 carbons. For instance, the non-sulfate based anionic surfactant may comprise sodium methyl cocoyl taurate, sodium methyl oleoyl taurate, or a combination thereof.

The amphoteric surfactant may be selected from alkyl amphopropionates, betaines, alkyl sultaines, alkyl amphoacetates, and a combination of two or more thereof. Preferably, the amphoteric surfactant comprises a betaine surfactant. In at least one embodiment, the amphoteric surfactant comprises cocamidopropyl betaine.

The nonionic surfactant may comprise a glucoside surfactant, a glyceride surfactant, or a combination of two or more thereof. Examples of glyceride surfactants include those selected from glycerol monostarate, glyceryl oleate, glyceryl linoleate, glyceryl ricinoleate, PEG glyceryl oleate, PCA glyceryl oleate, and a combination of two or more thereof. Examples of glucoside surfactant include those selected from lauryl glucoside, lauroyl ethyl glucoside, myristoyl ethyl glucoside, oleoyl ethyl glucoside, and a combination of two or more thereof. In at least one embodiment, the nonionic surfactant comprises PEG methyl glucose diolcate.

The polyol may comprise ethylene glycol, propylene glycol, butylene glycol, hexylene glycol, glycerin, diglycerin, diethylene glycol, and dipropylene glycol, or a combination of two more thereof. In some instances, the polyol comprises a polyethylene glycol, a polypropylene glycol, a block polymer of polyethylene glycol and polypropylene glycol, or a combination of two or more thereof.

The personal care composition may also include a preservative system comprising lactic acid, citric acid, sodium benzoate, sodium salicylate, tetrasodium N,N-bis(carboxymethyl)-L-glutamate, or a combination of two or more thereof. In at least one embodiment, the preservative system comprises lactic acid and/or a salt thereof. In further embodiment, the preservative system comprises citric acid and/or a salt thereof and salicylic and/or a salt thereof. The preservative system may comprise tetrasodium N,N-bis(carboxymethyl)-L-glutamate and at least one preservative selected from citric acid, salicylic, a salt thereof, and a combination of two or more thereof.

In some embodiments, the personal care composition has about 6 wt. % or less of sulfate based anionic surfactants. The personal care composition may be essentially free of or free of sulfate based anionic surfactants.

The personal care composition may have about 2 wt. % or less of ethylenediaminetetraacetic acid (EDTA). In some cases, the personal care composition is essentially free of or free of ethylenediaminetetraacetic acid (EDTA).

The personal care composition may be essentially free of or free of an acrylate based thickener. Additionally or alternatively, the personal care composition may be essentially free of or free of a polymer based thickener. In some cases, the personal care composition may be essentially free of or free of a polysaccharide based thickener.

In certain preferred embodiments, the personal care composition has a natural origin index of about 90% or more pursuant to ISO Standard 16128.

In further preferred embodiments, the personal care composition has a biodegradability index of about 85% or more pursuant to ISO Standard 14855-1:2005.

In accordance with another aspect, provided is a personal care composition comprising a surfactant system comprising PEG methyl glucose dioleate and one or more surfactant selected from: sulfonate surfactant, a taurate surfactant, a glyceride surfactant, or an alkyl glycoside surfactant; and a polyol.

The PEG methyl glucose dioleate, in some cases, may be PEG-120 methyl glucose dioleate. The personal care composition may have a pH of about 2 to about 4.5.

In accordance with a further aspect, provided is a personal care composition having a surfactant system comprising: a taurate surfactant, a betaine surfactant, and a glycoside surfactant; and a polyol, wherein the personal care composition has a natural origin index of about 90% or more pursuant to ISO Standard 16128 and a biodegradability index of about 85% or more pursuant to ISO Standard 14855-1:2005. The personal care composition may have a viscosity of about 3,000 cP or more.

A list of non-limiting, example embodiments is provided below.

In accordance with embodiment 1, provided is a personal care composition comprising: from about 1 to about 26 wt. % of a surfactant system comprising:
a non-sulfate anionic surfactant,
an amphoteric surfactant, and
a nonionic surfactant; and
a polyol,
wherein the personal care composition has a pH of about 2 to about 4.5, and all weight percentages are based on the total weight of the personal care composition.

In accordance with embodiment 2, provided is a personal care composition according to embodiment 1, wherein a weight ratio of non-sulfate based anionic surfactant to amphoteric surfactant from about 1:2 to about 1:9.

In accordance with embodiment 3, provided is a personal care composition according to embodiment 1 or embodiment 2, wherein the non-sulfate anionic surfactant is selected from a sulfonate surfactant, a taurate surfactant, an isethionate surfactant, a sarcosinate surfactant, a sulfonate surfactant, a sulfosuccinate surfactant, a sulfoacetate surfactant, an alkoxylated monoacid surfactant, a salt thereof, and a combination of two or more thereof.

In accordance with embodiment 4, provided is a personal care composition according to embodiment 3, wherein the non-sulfate based anionic surfactant comprises a taurate surfactant having an aliphatic carbon chain of 8 to 21 carbons.

In accordance with embodiment 5, provided is a personal care composition according to any foregoing embodiment, wherein the non-sulfate based anionic surfactant comprises sodium methyl cocoyl taurate, sodium methyl olcoyl taurate, or a combination thereof.

In accordance with embodiment 6, provided is a personal care composition according to any foregoing embodiment, wherein the amphoteric surfactant is selected from alkyl amphopropionates, betaines, alkyl sultaines, alkyl amphoacetates, and a combination of two or more thereof.

In accordance with embodiment 7, provided is a personal care composition according to any foregoing embodiment, wherein the amphoteric surfactant comprises a betaine surfactant.

In accordance with embodiment 8, provided is a personal care composition according to any foregoing embodiment, wherein the amphoteric surfactant comprises cocamidopropyl betaine.

In accordance with embodiment 9, provided is a personal care composition according to any foregoing embodiment, wherein the nonionic surfactant comprises a glucoside surfactant, a glyceride surfactant, or a combination of two or more thereof.

In accordance with embodiment 10, provided is a personal care composition according to embodiment 9, wherein the glyceride surfactant is selected from glycerol monostarate, glyceryl oleate, glyceryl linoleate, glyceryl ricinoleate, PEG glyceryl oleate, PCA glyceryl oleate, and a combination of two or more thereof.

In accordance with embodiment 11, provided is a personal care composition according to embodiment 9, wherein the glucoside surfactant is selected from lauryl glucoside, lauroyl ethyl glucoside, myristoyl ethyl glucoside, oleoyl ethyl glucoside, and a combination of two or more thereof.

In accordance with embodiment 12, provided is a personal care composition according to one of embodiments 9 to 11, wherein the nonionic surfactant comprises PEG methyl glucose dioleate.

In accordance with embodiment 13, provided is a personal care composition according to any foregoing embodiment, wherein the surfactant system is present in an amount from about 1 to about 20 wt. %.

In accordance with embodiment 14, provided is a personal care composition according to any foregoing embodiment, wherein the non-sulfate anionic surfactant is present in an amount from about 0.3 to about 10 wt. %.

In accordance with embodiment 15, provided is a personal care composition according to any foregoing embodiment, wherein the amphoteric surfactant is present in an amount from about 1 to about 9 wt. %.

In accordance with embodiment 16, provided is a personal care composition according to any foregoing embodiment, wherein the nonionic surfactant is present in an amount from about 1 to about 11 wt. %.

In accordance with embodiment 17, provided is a personal care composition according to any foregoing embodiment, wherein the polyol comprises ethylene glycol, propylene glycol, butylene glycol, hexylene glycol, glycerin, diglycerin, diethylene glycol, and dipropylene glycol, or a combination of two more thereof.

In accordance with embodiment 18, provided is a personal care composition according to any foregoing embodiment, wherein the polyol comprises a polyethylene glycol, a polypropylene glycol, a block polymer of polyethylene glycol and polypropylene glycol, or a combination of two or more thereof.

In accordance with embodiment 19, provided is a personal care composition according to any foregoing embodiment further comprising a preservative system comprising lactic acid, citric acid, sodium benzoate, sodium salicylate, tetrasodium N,N-bis(carboxymethyl)-L-glutamate, or a combination of two or more thereof.

In accordance with embodiment 20, provided is a personal care composition according to embodiment 19, wherein the preservative system comprises lactic acid and/or a salt thereof.

In accordance with embodiment 21, provided is a personal care composition according to embodiment 19 or 20, wherein the preservative system comprises citric acid and/or a salt thereof and salicylic and/or a salt thereof.

In accordance with embodiment 22, provided is a personal care composition according to one of embodiments 19 to 21, wherein the preservative system comprises tetrasodium N,N-bis(carboxymethyl)-L-glutamate and at least one preservative selected from citric acid, salicylic, a salt thereof, and a combination of two or more thereof.

In accordance with embodiment 23, provided is a personal care composition according to any foregoing embodiment, wherein the personal care composition has about 6 wt. % or less of sulfate based anionic surfactants.

In accordance with embodiment 24, provided is a personal care composition according to any foregoing embodiment, wherein the personal care composition is essentially free of or free of sulfate based anionic surfactants.

In accordance with embodiment 25, provided is a personal care composition according to any foregoing embodiment, wherein the personal care composition has about 2 wt. % or less of ethylenediaminetetraacetic acid (EDTA).

In accordance with embodiment 26, provided is a personal care composition according to any foregoing embodiment, wherein the personal care composition is essentially free of or free of ethylenediaminetetraacetic acid (EDTA).

In accordance with embodiment 27, provided is a personal care composition according to any foregoing embodiment, wherein the personal care composition has a natural origin index of about 90% or more pursuant to ISO Standard 16128.

In accordance with embodiment 28, provided is a personal care composition according to any foregoing embodiment, wherein the personal care composition has a biodegradability index of about 85% or more pursuant to ISO Standard 14855-1:2005.

In accordance with embodiment 29, provided is a personal care composition comprising:
a surfactant system comprising PEG methyl glucose dioleate and one or more surfactant selected from:
   a sulfonate surfactant,
   a taurate surfactant,
   a glyceride surfactant, or
   a glycoside surfactant; and
a polyol.

In accordance with embodiment 30, provided is a personal care composition according to embodiment 29, wherein the PEG methyl glucose dioleate comprises PEG-120 methyl glucose dioleate.

In accordance with embodiment 31, provided is a personal care composition according to embodiment 29 or 30, wherein the personal care composition has a pH of about 2 to about 4.5.

In accordance with embodiment 32, provided is a personal care composition according to one of embodiments 29 to 31, wherein the personal care composition has a natural origin index of about 90% or more pursuant to ISO Standard 16128.

In accordance with embodiment 33, provided is a personal care composition according to one of embodiments 29 to 32, wherein the personal care composition has a biodegradability index of about 85% or more pursuant to ISO Standard 14855-1:2005.

In accordance with embodiment 34, provided is a personal care composition comprising:
a surfactant system comprising:
   a taurate surfactant,
   a betaine surfactant, and
   a glycoside surfactant;
a polyol,
wherein the personal care composition has a natural origin index of about 90% or more pursuant to ISO Standard 16128 and a biodegradability index of about 85% or more pursuant to ISO Standard 14855-1:2005.

In accordance with embodiment 35, provided is a personal care composition according to embodiment 24, wherein the personal care composition has a pH of about 2 to about 4.5.

In accordance with embodiment 36, provided is a personal care composition according to any foregoing embodiment, wherein the personal care composition has a viscosity of about 3,000 cP or more.

In accordance with embodiment 37, provided is a personal care composition according to embodiment 36, wherein the personal care composition is essentially free of or free of an acrylate based thickener.

In accordance with embodiment 38, provided is a personal care composition according to embodiment 36 or 37, wherein the personal care composition is essentially free of or free of a polymer based thickener.

In accordance with embodiment 39, provided is a personal care composition according to one of embodiments 36 to 38, wherein the personal care composition is essentially free of or free of a polysaccharide based thickener.

In accordance with embodiment 40, provided is a personal care composition according to one of embodiments 36 to 38, wherein the personal care composition has a weight ratio of the taurate surfactant to betaine surfactant from about 1:2 to about 1:9.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other apparatuses and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the present disclosure can be free or essentially free of all components and elements positively recited throughout the present disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both a surfactant and an emulsifier. If a particular personal care composition includes both a surfactant and an emulsifier, a compound that may be characterized as both a surfactant and an emulsifier will serve only as either a surfactant or an emulsifier—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to personal care compositions and particularly personal care compositions having a preferable natural origin index and/or a preferable biodegradability index. The inventors unexpectedly discovered that personal care compositions having surfactant systems comprising certain surfactants in particular amounts and ratios disclosed herein with certain pHs can achieve preferable viscosities while simultaneously having a preferable natural origin index and/or biodegradability index. In some embodiments, the personal care compositions can achieve a desirable viscosity while having a minimal amount or being free of traditional thickening agents, such as an acrylate based thickener, a polymer based thickener, and/or a polysaccharide based thickener.

In accordance with one aspect, provided is a personal care composition having from about 1 to about 26 wt. % a surfactant system comprising a non-sulfate anionic surfactant, an amphoteric surfactant, and a nonionic surfactant; and a polyol, wherein the personal care composition has a pH of about 2 to about 4.5, and all weight percentages are based on the total weight of the personal care composition.

The personal care compositions may have a natural origin index of about 90% or more, e.g., as measured pursuant to ISO Standard 16128. For example, in some embodiments, the personal care product may have a natural origin index pursuant to ISO Standard 16128 of about 90% or more, about 91% or more, about 92% or more, about 93% or more, about 94% or more, about 95% or more, about 96% or more, about 97% or more, about 98% or more, about 99% or more, or about 99.5% or more.

The personal care compositions may, additionally or alternatively, achieve a biodegradability index of about 85% or more, e.g., as measured pursuant to ISO Standard 14855-1:2005. In some instances, the personal care compositions may have a biodegradability index pursuant to ISO Standard 14855-1:2005 of about 85% or more, about 87% or more, about 88% or more, about 89% or more, about 90% or more, about 91% or more, about 92% or more, about 93% or more, about 94% or more, about 95% or more, about 96% or more, about 97% or more, about 98% or more, about 99% or more, or about 99.5% or more.

In some embodiments, the personal care compositions are essentially free of or free of sulfate based anionic surfactants. For example, the personal care compositions may be formulated to have about 6 wt. % or less, about 4 wt. % or less, about 3 wt. % or less, about 2 wt. % or less, about 1 wt. % or less, about 0.5 wt. % or less, or about 0.1 wt. % or less, of sulfate based anionic surfactants, based on the total weight of the personal care composition. The personal care composition is free of sulfate based anionic surfactants in at least one embodiment.

The personal are compositions may also be formulated to be essentially free of or free of ethylenediaminetetraacetic acid (EDTA). In some instances, the personal care compositions have EDTA in an amount of about 3 wt. % or less, about 2 wt. % or less, about 1 wt. % or less, about 0.5 wt. % or less, about 0.1 wt. % or less, or about 0.05 wt. % or less, based on the total weight of the personal care composition of EDTA. In at least one embodiment, the personal care composition is free of EDTA.

The personal care compositions preferably have less than about 1 ppm of 1,4 dioxane. In some embodiments, the personal care compositions have 1 ppm or less, about 0.7 ppm or less, about 0.5 ppm or less, about 0.3 ppm or less, or about 0.1 ppm or less of 1,4 dioxane.

The personal care compositions may be formulated to have a viscosity of about 3,000 to about 15,000 cP using a Brookfield DV2T viscometer with a spindle number 5 at 20 RPM and a temperature of 25° C. in a non-diluted form. In some instances, the personal care composition has a viscosity of from about 3,000 to about 15,000 cP, about 3,000 to about 12,000 cP, about 3,000 to about 10,000 cP, about 3,000 to about 8,000 cP, about 3,000 to about 7,000 cP, about 3,000 to about 6,000 cP; from about 3,500 to about 15,000 cP, about 3,500 to about 12,000 cP, about 3,500 to about 10,000 cP, about 3,500 to about 8,000 cP, about 3,500 to about 7,000 cP, about 3,500 to about 6,000 cP; from about 4,000 to about 15,000 cP, about 4,000 to about 12,000 cP, about 4,000 to about 10,000 cP, about 4,000 to about 8,000 cP, about 4,000 to about 7,000 cP, about 4,000 to about 6,000 cP; from about 4,500 to about 15,000 cP, about 4,500 to about 12,000 cP, about 4,500 to about 10,000 cP, about 4,500 to about 8,000 cP, about 4,500 to about 7,000 cP, about 4,500 to about 6,000 cP, or any range or subrange thereof using a Brookfield DV2T viscometer with a spindle number 5 at 20 RPM and a temperature of 25° C.

Suitable components, such as those listed below, may be included or excluded from the formulations for the personal care compositions depending on the specific combination of other components, the form of the personal care compositions, and/or the use of the formulation (e.g., a ready-to-use personal care composition, a body wash composition, a cleansing composition, an antibacterial soap, etc.).

The personal care compositions include a surfactant system comprising two or more surfactants. The surfactant system typically includes two or more surfactants selected from anionic surfactants (e.g., sulfate based and/or non-sulfate based anionic surfactants), amphoteric surfactants, nonionic surfactants, and combinations of two or more thereof. For example, the surfactant system may include two, three, four, five, six, seven, eight, or nine or more surfactants. In some embodiments, the surfactant system comprises from two to eight, from two to seven, from two to six, from two to five, from two to four, from three to seven, from three to six, from three to five, from four to eight, four to seven, four to six, or any range or subrange thereof. Preferably, the surfactant system may include two or more surfactants, with the two or more surfactants being chosen from different categories of non-sulfate anionic surfactants, amphoteric surfactants, and nonionic surfactants. For example, the surfactant system may comprise at least one non-sulfate anionic surfactant, at least one amphoteric surfactant, and at least one nonionic surfactant. In some embodiments, the surfactant system consists of at least one anionic surfactant (e.g., a sulfate based surfactant and/or non-sulfate based surfactant), at least one amphoteric surfactant, and at least one nonionic surfactant. In further embodiments, the surfactant system consists of at least one non-sulfate anionic surfactant, at least one amphoteric surfactant (e.g., a betaine surfactant), and at least one nonionic surfactant (e.g., a glucoside surfactant).

The surfactant system may be present in the personal care composition in an amount ranging from about 1 to about 26 wt. %, based on the total weight of the personal care composition. For example, the surfactant system may be present in the personal are composition in an amount ranging from about 1 to about 23 wt. %, about 1 to about 20 wt. %, about 1 to about 18 wt. %, about 1 to about 16 wt. %, about 1 to about 14 wt. %, about 1 to about 12 wt. %, about 1 to about 10 wt. %; from about 3 to about 26 wt. %, about 3 to about 23 wt. %, about 3 to about 20 wt. %, about 3 to about 18 wt. %, about 3 to about 16 wt. %, about 3 to about 14 wt. %, about 3 to about 12 wt. %, about 3 to about 10 wt. %; from about 5 to about 26 wt. %, about 5 to about 23 wt. %, about 5 to about 20 wt. %, about 5 to about 18 wt. %, about 5 to about 16 wt. %, about 5 to about 14 wt. %, about 5 to about 12 wt. %, about 5 to about 10 wt. %; from about 7 to about 26 wt. %, about 7 to about 23 wt. %, about 7 to about 20 wt. %, about 7 to about 18 wt. %, about 7 to about 16 wt. %, about 7 to about 14 wt. %, about 7 to about 12 wt. %, about 7 to about 10 wt. %; from about 9 to about 26 wt. %, about 9 to about 23 wt. %, about 9 to about 20 wt. %, about 9 to about 18 wt. %, about 9 to about 16 wt. %, about 9 to about 14 wt. %, about 9 to about 12 wt. %; from about 11 to about 26 wt. %, about 11 to about 23 wt. %, about 11 to about 20 wt. %, about 11 to about 18 wt. %, about 11 to about 16 wt. %, about 11 to about 14 wt. %, or any range or subrange thereof, based on the total weight of the personal care composition.

The anionic surfactants may be selected for sulfate based surfactants, such as a sulfate based anionic surfactants selected from ammonium lauryl sulfate, ammonium lauryl ether sulfate, sodium dodecyl sulfate, sodium coco-sulfate, ammonium coco-sulfate, and a combination of two or more thereof. Additionally or alternatively, the anionic surfactant may comprise or consist of non-sulfate based anionic surfactants, such as those selected from sulfonate surfactants, taurate surfactants, isethionate surfactants, sarcosinate surfactants, sulfonate surfactants, sulfosuccinate surfactants, sulfoacetate surfactants, alkoxylated monoacid surfactants, salts thereof, and combinations of two or more thereof. The anionic surfactant may be present in the personal care composition in an amount from about 0.3 to about 10 wt. %, based on the total weight of the personal care composition. For instance, the personal care composition may include non-sulfate based anionic surfactants in an amount ranging from about 0.3 to about 8 wt. %, about 0.3 to about 6 wt. %, about 0.3 to about 4 wt. %, about 0.3 to about 3 wt. %, about 0.3 to about 2 wt. %; from about 0.5 to about 10 wt. %, about 0.5 to about 8 wt. %, about 0.5 to about 6 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 4 wt. %, about 1 to about 3 wt. %, about 1 to about 2 wt. %; from about 1.5 to about 10 wt. %, about 1.5 to about 8 wt. %, about 1.5 to about 6 wt. %, about 1.5 to about 4 wt. %, about 1.5 to about 3 wt. %; from about 2 to about 10 wt. %, about 2 to about 8 wt. %, about 2 to about 6 wt. %, about 2 to about 4 wt. %, about 2 to about 3 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %; from about 5 to about 10 wt. %, about 5 to about 8 wt. %, about 5 to about 6 wt. %, or any range or subrange thereof, based on the total weight of the personal care composition. The non-sulfate based anionic surfactant may have an alkyl and/or acyl group comprising 6 to 26 carbon atoms, 8 to 26 carbon atoms, 8 to 21 carbons, 8 to 16 carbon atoms, 10 to 16 carbon atoms, or 10 to 13 carbon atoms. In some instances, the non-sulfate based anionic surfactant comprises an alkyl and/or acyl group has more than 26 carbons.

The anionic surfactant may be selected from taurate surfactants having a structure according to the following formula:

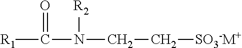

wherein R₁ is a saturated or unsaturated, straight or branched alkyl chain with 6 to 18 carbon atoms, R₂ is H or methyl, and M⁺ is H, sodium, or potassium (e.g., sodium methyl cocoyl taurate).

Additional examples of taurate surfactants include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations of two or more thereof. In some embodiments, the personal care composition comprises sodium lauroyl methyl taurate (or sodium methyl lauroyl taurate), sodium methyl cocoyl taurate (SMCT), or a combination thereof. In at least one preferred embodiment, the surfactant system comprises sodium methyl cocoyl taurate.

Non-limiting examples of isethionate surfactants include sodium isethionate, sodium cocoyl isethionate, sodium lauroyl methyl isethionate, and sodium cocoyl methyl isethionate. Sulfonated monoglycerides of fatty acids include sodium coconut monoglyceride sulfonates and the like. Disodium 2-sulfolaurate is an additional sulfonate surfactant that may be included in certain embodiments of the personal care composition. Examples of sarcosinate surfactants include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations of two or more thereof. Non-limiting examples of alkyl taurate salts include sodium cocoyl taurate, sodium methyl cocoyl taurate, oleoyl taurate. In at least one preferred embodiment, the personal care composition includes one or more anionic surfactant comprising oleoyl taurate and/or a salt thereof.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a combination of two or more thereof.

Non-limiting examples of alkyl sulfacetates includes, e.g., alkyl sulfoacetates such as C₄-C₁₈ fatty alcohol sulfoacetates and/or salts thereof. A preferred sulfoacetate salt is sodium lauryl sulfoacetate. Useful cations for the salts include alkali metal ions such as sodium or potassium, ammonium ions, or alkanolammonium ions such as monoethanolammonium 1 or triethanolammonium ions.

Examples of alkoxylated monoacids include, but are not limited to: butoxynol-5 carboxylic acid, butoxynol-19 carboxylic acid, capryleth-4 carboxylic acid, capryleth-6 carboxylic acid, capryleth-9 carboxylic acid, ceteareth-25 carboxylic acid, coceth-7 carboxylic acid, C⁹⁻¹¹ pareth-6 carboxylic acid, C₁₁₋₁₅ pareth-7 carboxylic acid, C₁₂₋₁₃ pareth-5 carboxylic acid, C₁₂₋₁₃ pareth-8 carboxylic acid, C₁₂₋₁₃ pareth-12 carboxylic acid, C₁₂₋₁₅ pareth-7 carboxylic acid, C₁₂₋₁₅ pareth-8 carboxylic acid, C₁₄₋₁₅ pareth-8 carboxylic acid, deceth-7 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, PPG-6-laureth-6 carboxylic acid, ppg-8-steareth-7 carboxylic acid, myreth-3 carboxylic acid, myreth-5 carboxylic acid, nonoxynol-5 carboxylic acid, nonoxynol-8 carboxylic acid, nonoxynol-10 carboxylic acid, octeth-3 carboxylic acid, octoxynol-20 carboxylic acid, oleth-3 carboxylic acid, oleth-6 carboxylic acid, oleth-10 carboxylic acid, ppg-3-deceth-2 carboxylic acid, capryleth-2 carboxylic acid, ceteth-13 carboxylic acid, deceth-2 carboxylic acid, hexeth-4 carboxylic acid, isosteareth-6 carboxylic acid, isosteareth-11 carboxylic acid, trudeceth-3 carboxylic acid, trideceth-6 carboxylic acid, trideceth-8 carboxylic acid, trideceth-12 carboxylic acid, trideceth-3 carboxylic acid, trideceth-4 carboxylic acid, trideceth-7 carboxylic acid, trideceth-15 carboxylic acid, trideceth-19 carboxylic acid, undeceth-5 carboxylic acid and a combination of two or more thereof. In some embodiments, one or more of the foregoing anionic surfactants are excluded from the personal care composition. For instance, in some embodiments, the surfactant system comprises at least one anionic surfactant, wherein the anionic surfactant(s) consists of taurate surfactant(s) and/or sulfonate anionic surfactant (such as, disodium 2-sulfolaurate).

The surfactant system may include one or more amphoteric surfactant(s) in an amount that may vary, but typically ranges from about 1 to about 9 wt. %, based on the total weight of the personal care composition. For example, the total amount of amphoteric surfactant(s) in the personal care composition may be from about 1 to about 7 wt. %, about 1 to about 6 wt. %, about 1 to about 5 wt. %, about 1 to about 4 wt. %; from about 2 to about 9 wt. %, about 2 to about 7 wt. %, about 2 to about 6 wt. %, about 2 to about 5 wt. %, about 2 to about 4 wt. %; from about 3 to about 9 wt. %, about 3 to about 7 wt. %, about 3 to about 6 wt. %, about 3 to about 5 wt. %; from about 4 to about 9 wt. %, about 4 to about 7 wt. %, about 4 to about 6 wt. %, about 4 to about 5 wt. %; from about 5 to about 9 wt. %, about 5 to about 7 wt. %, or any range or subrange thereof, based on the total weight of the personal care composition.

Amphoteric surfactants are typically characterized by a combination of high surfactant activity, lather forming and mildness. The amphoteric surfactant may comprise a substituent, such as an alkyl or acyl group, containing 8 to 18 carbon atoms and/or a substituent containing one or more carboxylate, sulfonate, sulfate, phosphate, or phosphonate. For instance, the amphoteric surfactant may have an alkyl group comprising from 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxy, sulfonate, sulfate, phosphate, or phosphonate.

The amphoteric surfactants may include amphoproprionate surfactants, betaine surfactants, sultaine surfactants, alkyl amphoacetate surfactants, and a combination of two or more thereof. In some instances, the personal care composition includes one or more alkyl amphopropionates. Non-limiting examples of alkyl amphopropionate surfactants include cocoamphopropionate, cornamphopropionate, caprylamphopropionate, cornamphopropionate, caproamphopropionate, oleoamphopropionate, isostearoamphopropionate, stearoamphopropionate, lauroamphopropionate, salts thereof, and a mixture thereof. In one embodiment, the alkyl amphopropionate surfactant comprises sodium cocoamphopropionate.

The personal care composition may include an amphoteric surfactant selected from betaine surfactants (also referred to as betaines). Examples of betaine surfactants include, but are not limited to high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl) carboxy methyl betaine, stearyl bis-(2-hydroxypropyl) carboxymethyl betaine, oleyl dimethyl gamma-carboxypropyl betaine, and lauryl bis-(2-hydroxypropyl) alpha-carboxyethyl betaine; sulfobetaines, such as coco dimethyl sulfopropyl betaine, stearyl dimethyl sulfopropyl betaine; amido betaines; amidosulfobetaines; and the like. Preferably, a betaine surfactant is selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and combinations of two or more thereof. In some embodiments, at least one betaine surfactant is selected from coco betaine, cocamidopropyl betaine, behenyl betaine, capryl/capramidopropyl betaine, and lauryl betaine, and a combination of two or more thereof. In further embodiments, the betaine surfactant is selected from cocamidopropyl betaine, lauryl dimethyl betaine, cetyl dimethyl betaine, and cocoamphodiacetate, and a combination of two or more thereof. In at least one embodiment, the personal care composition comprises coco betaine, cocamidopropyl betaine, or a combination thereof.

The amphoteric surfactant may, in some cases, be selected sultaines, also known as sulfobetaines, and hydroxy sultaines, for example, cocamidopropyl hydroxysultain. The sultaines may be an alkyl sultaines having from 6 to 20 carbons, 8 to 18 carbons, 8 to 16 carbons, or 10 to 16 carbons. Non-limiting examples of alkyl sultaines include hydroxyl sultaines having a structure according to the following formula:

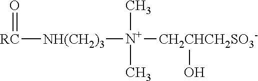

wherein R is an alkyl group having 8-18 carbon atoms. Examples of alkyl sultaine surfactants include, but are not limited to, cocamidopropyl hydroxysultaine, lauryl hydroxysultaine, and a combination of two or more thereof.

In some embodiments, the surfactant system includes an amphoteric surfactant selected from betaine surfactants and, optionally, an additional amphoteric surfactant other than betaines including those discussed above. The one or more betaine surfactants may, preferably, be selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and a combination of two or more thereof. In some embodiments, one or more of the foregoing amphoteric surfactants are excluded from the personal care composition. For instance, the surfactant system may include at least one amphoteric surfactant(s), where the amphoteric surfactant(s) consist of betaine surfactant(s).

The surfactant system may comprise one or more nonionic surfactant(s). The one or more nonionic surfactant(s) may be present in an amount from about 1 to about 11 wt. %, based on the total weight of the personal care composition. In some instances, the one or more nonionic surfactant(s) is present in the surfactant system in an amount from about 1 to about 9 wt. %, about 1 to about 7 wt. %, about 1 to about 6 wt. %, about 1 to about 5 wt. %, about 1 to about 4 wt. %; from about 2 to about 11 wt. %, about 2 to about 9 wt. %, about 2 to about 7 wt. %, about 2 to about 6 wt. %, about 2 to about 5 wt. %, about 2 to about 4 wt. %; from about 3 to about 11 wt. %, about 3 to about 9 wt. %, about 3 to about 7 wt. %, about 3 to about 6 wt. %, about 3 to about 5 wt. %; from about 4 to about 11 wt. %, about 4 to about 9 wt. %, about 4 to about 7 wt. %, about 4 to about 6 wt. %; from about 5 to about 11 wt. %, about 5 to about 9 wt. %, about 5 to about 7 wt. %; from about 6 to about 11 wt. %, about 6 to about 9 wt. %, about 7 to about 11 wt. %, or any range or subrange thereof, based on the total weight of the personal care composition.

The one or more nonionic surfactant(s) may be selected from glycoside surfactants, glucoside surfactants, glyceride surfactants, compounds produced by the condensation of alkylene oxide groups (hydrophilic in nature) with an organic hydrophobic compound, which may be aliphatic or alkylaromatic in nature, and combinations of two or more thereof. Preferably, at least one nonionic surfactant is selected from a glycoside surfactant, a glucoside surfactant, a glyceride surfactant, and a combination of two or more thereof. In some embodiments, the personal care composition comprises two or more (e.g., 2, 3, 4, or 5) nonionic surfactants. In some embodiments, the nonionic surfactant(s) consist of one or more glycoside surfactant, one or more glucoside surfactant, one or more glyceride surfactant, and a combination of two or more thereof. The nonionic surfactant(s) consist of glucoside surfactant(s) in at least one embodiment.

The surfactant system may comprise one or more glyceride surfactant selected from glycerol monostarate, glyceryl olcate, glyceryl linolcate, glyceryl ricinoleate, PEG glyceryl olcate, PCA glyceryl oleate, and a combination of two or more thereof. In at least one embodiment, the PEG glyceryl oleate may be PEG-18 glyceryl oleate. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. In some embodiments, the surfactant system includes one or more nonionic surfactant(s) chosen from lauryl glucoside, lauroyl ethyl glucoside, myristoyl ethyl glucoside, oleoyl ethyl glucoside, and a combination of two or more thereof. Additionally or alternatively, the glucoside surfactant may be chosen from polyglucosides, such as alkylpolyglucosides.

Further examples of nonionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials. Additional examples of nonionic surfactants include polyoxyethylene, polyoxyethylene sorbitan esters, polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, or a combination of two or more thereof. In some instances, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, or a combination of two or more thereof.

In at least one preferred embodiment, the nonionic surfactant comprises PEG methyl glucose dioleate, coco-glucoside, or a combination thereof. The PEG methyl glucose dioleate may be PEG-120 methyl glucose dioleate.

The personal care composition may be formulated to have a surfactant system having certain weight ratios of the total amount of non-sulfate based anionic surfactant(s) to the total amount of amphoteric surfactant(s). For example, in some preferred embodiments, the personal care composition has a weight ratio of non-sulfate based anionic surfactant(s) to amphoteric surfactant(s) ranging from about 1:2 to about 1:9, about 1:2 to about 1:8, about 1:2 to about 1:7, about 1:2 to about 1:6.5, about 1:2 to about 1:6; from about 1:3 to about 1:9, about 1:3 to about 1:8, about 1:3 to about 1:7, about 1:3 to about 1:6.5, about 1:3 to about 1:6; from about 1:3.5 to about 1:9, about 1:3.5 to about 1:8, about 1:3.5 to about 1:7, about 1:3.5 to about 1:6.5, about 1:3.5 to about 1:6; about 1:4 to about 1:9, about 1:4 to about 1:8, about 1:4 to about 1:7, about 1:4 to about 1:6.5, about 1:4 to about 1:6, or any range or subrange thereof.

Additionally or alternatively, the surfactant system may have a weight ratio of the total amount of taurate surfactant(s) (e.g., sodium methyl cocoyl taurate and/or sodium methyl oleoyl taurate) to the total amount of betaine surfactant(s) (e.g., cocamidopropyl betaine) ranging from about 1:2 to about 1:9. For instance, the surfactant system may have a weight ratio of the taurate surfactant(s) to betaine surfactant(s) ranging about 1:2 to about 1:8, about 1:2 to about 1:7, about 1:2 to about 1:6.5, about 1:2 to about 1:6; from about 1:3 to about 1:9, about 1:3 to about 1:8, about 1:3 to about 1:7, about 1:3 to about 1:6.5, about 1:3 to about 1:6; from about 1:3.5 to about 1:9, about 1:3.5 to about 1:8, about 1:3.5 to about 1:7, about 1:3.5 to about 1:6.5, about 1:3.5 to about 1:6; about 1:4 to about 1:9, about 1:4 to about 1:8, about 1:4 to about 1:7, about 1:4 to about 1:6.5, about 1:4 to about 1:6, or any range or subrange thereof.

The personal care composition may be formulated to have a surfactant system having certain weight ratios of the total amount of nonionic surfactant(s) to the total amount of amphoteric surfactant(s). For example, the surfactant system may have a weight ratio of the total amount of nonionic surfactant to amphoteric surfactant ranging from about 1.5:1 to about 3.5:1. In some embodiments, the personal care composition has a weight ratio of the total amount of nonionic surfactant to amphoteric surfactant ranging from about 1.5:1 to about 3.5:1, about 2:1 to about 3.5:1, about 2.5:1 to about 3.5:1, about 3:1 to about 3.5:1; from about 1.5:1 to about 3:1, about 2:1 to about 3:1, about 2.5:1 to about 3:1, about 3:1 to about 3:1; from about 1.5:1 to about 2.5:1, about 2:1 to about 2.5:1, or any range or subrange thereof.

Additionally or alternatively, the personal care composition may be formulated to have a surfactant system having certain weight ratios for the total amount of glucoside surfactant(s) to the total amount of betaine surfactant(s). For example, the surfactant system may have a weight ratio of the total amount of nonionic surfactant to amphoteric surfactant ranging from about 1.5:1 to about 3.5:1. In some embodiments, the personal care composition has a weight ratio of the total amount of nonionic surfactant to amphoteric surfactant ranging from about 1.5:1 to about 3.5:1, about 2:1 to about 3.5:1, about 2.5:1 to about 3.5:1, about 3:1 to about 3.5:1; from about 1.5:1 to about 3:1, about 2:1 to about 3:1, about 2.5:1 to about 3:1, about 3:1 to about 3:1; from about 1.5:1 to about 2.5:1, about 2:1 to about 2.5:1, or any range or subrange thereof.

The personal care composition may include one or more polyol(s). Preferably, the personal care compositions include the one or more polyol(s) in an amount from about 1 to about 25 wt. %, based on the total weight of the personal care composition. For example, the total amount of polyol(s) in the personal care composition may be from about 1 to about 22 wt. %, about 1 to about 18 wt. %, about 1 to about 12 wt. %, about 1 to about 8 wt. %, about 1 to about 6 wt. %, about 1 to about 4 wt. %, about 1 to about 3 wt. %, about 1 to about 22 wt. %; from about 2 to about 22 wt. %, about 2 to about 18 wt. %, about 2 to about 12 wt. %, about 2 to about 8 wt. %, about 2 to about 6 wt. %, about 2 to about 4 wt. %, about 2 to about 3 wt. %; from about 3 to about 22 wt. %, about 3 to about 18 wt. %, about 3 to about 12 wt. %, about 3 to about 8 wt. %, about 3 to about 6 wt. %; from about 5 to about 25 wt. %, about 5 to about 22 wt. %, about 5 to about 20 wt. %, about 5 to about 18 wt. %, about 5 to about 16 wt. %, about 5 to about 12 wt. %, about 5 to about 10 wt. %, about 5 to about 8 wt. %; from about 7 to about 25 wt. %, about 7 to about 22 wt. %, about 7 to about 20 wt. %, about 7 to about 18 wt. %, about 7 to about 16 wt. %; from about 9 to about 25 wt. %, about 9 to about 22 wt. %, about 9 to about 20 wt. %, about 9 to about 18 wt. %, about 9 to about 16 wt. %; from about 12 to about 25 wt. %, about 12 to about 22 wt. %, about 12 to about 20 wt. %, about 12 to about 18 wt. %, about 12 to about 16 wt. %; from about 14 to about 25 wt. %, about 14 to about 22 wt. %, about 14 to about 20 wt. %, about 14 to about 18 wt. %; from about 16 to about 25 wt. %, about 16 to about 22 wt. %, about 16 to about 20 wt. %; from about 18 to about 25 wt. %, about 21 to about 25 wt. %, or any range or subrange thereof, based on the total weight of the personal care composition.

The polyol(s) may be chosen from glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Polyols that may be included in the personal care composition, in certain instances, include ethylene glycol, propylene glycol, butylene glycol, hexylene glycol, glycerin, diglycerin, diethylene glycol, dipropylene glycol, and mixtures thereof. In some cases, the personal care composition comprises glycerin, and optionally one or more polyols other than glycerin. Non-limiting examples of polyols that may, optionally, be included in the personal care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include or be chosen from ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, and/or a mixture of two or more thereof. In certain embodiments, the personal care composition may include polyol(s) selected from glycerin (preferably, vegetable refined glycerin), butylene glycol, and 1,3-propanediol, polyglutamic acid, saccharide isomerate, and combinations thereof. In some embodiments, the polyol is a polyethylene glycol, such as polyethylene glycol 600.

Additionally or alternatively, the personal care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof.

The personal care composition may optionally include one or more thickening agent(s). The amount of thickening agent in the personal care composition, if present, may be from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %, or any range or subrange thereof, based on the total weight of the personal care composition.

Without being limited to any particular theory, the personal care compositions in some embodiments achieve preferable viscosities with a minimal amount or while being essentially free of or free of thickening agents (e.g., thickening agents other than surfactants). For example, the personal care composition may include thickening agent(s) in a total amount from about 0.1 wt. %, from about 0.5 wt. %, from about 1 wt. %, from about 2 wt. %, from about 3 wt. %, or from about 3.5 wt. % up to about 1 wt. %, up to about 2 wt. %, up to about 3 wt. %, up to about 4 wt. %, or up to about 5 wt. %, including any range or subrange formed therefrom, based on the total weight of the personal care composition. In some embodiments, the personal care composition may have thickening agent(s) in a total amount of about 5 wt. % or less, about 4 wt. % or less, about 3 wt. % or less, about 2 wt. % or less, about 1 wt. % or less, about 0.5 wt. % or less, or about 0.1 wt. % or less, based on the total weight of the personal care composition. In some preferred embodiments, the personal care composition is free of thickening agents except for those constituting surfactants.

Thickening agents may be referred to as “thickeners” or “viscosity modifying agents.” Thickening agents are typically included to increase the viscosity of the personal care compositions. Non-limiting examples of thickening agents that may be incorporated into or excluded from the personal care compositions include natural gums, polyacrylate crosspolymers or cross linked polyacrylate polymers, cationic acrylate copolymers, anionic acrylic or carboxylic acid polymers, polyacrylamide polymers, polysaccharides such as cellulose derivatives, cationic gums, polyquaterniums, vinylpyrrolidone homopolymers/copolymers, C₈₋₂₄ hydroxyl substituted aliphatic acid, C₈₋₂₄ conjugated aliphatic acid, sugar fatty esters, polyglyceryl esters, and a mixture thereof. Particular types of thickening agents that may be mentioned include the following: gums (e.g., natural gums), sucrose esters, polyvinylpyrrolidone (PVP) and co-polymers, celluloses, polyquaternium compounds, and carboxylic acid or carboxylate based homopolymer or co-polymer, which can be linear or crosslinked. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, carrageenan, or a combination of two more thereof. Examples of natural gums include karaya gum, gum arabic, and tragacanth gum.

In some embodiments, the personal care compositions are essentially free of or free of acrylate based thickener(s). For example, the personal care compositions may be formulated to have about 6 wt. % or less, about 4 wt. % or less, about 3 wt. % or less, about 2 wt. % or less, about 1 wt. % or less, about 0.5 wt. % or less, or about 0.1 wt. % or less, of acrylate based thickener, based on the total weight of the personal care composition. The personal care composition is free of acrylate based thickener in at least one embodiment.

Additionally or alternatively, the personal care compositions may be essentially free of or free of polymer based thickener(s). For example, the personal care compositions may be formulated to have about 6 wt. % or less, about 4 wt. % or less, about 3 wt. % or less, about 2 wt. % or less, about 1 wt. % or less, about 0.5 wt. % or less, or about 0.1 wt. % or less, of polymer based thickener, based on the total weight of the personal care composition. The personal care composition is free of polymer based thickener in at least one embodiment.

The personal care compositions may also be essentially free of or free of polysaccharide based thickener(s). For example, the personal care compositions may be formulated to have about 6 wt. % or less, about 4 wt. % or less, about 3 wt. % or less, about 2 wt. % or less, about 1 wt. % or less, about 0.5 wt. % or less, or about 0.1 wt. % or less, of polysaccharide based thickener, based on the total weight of the personal care composition. The personal care composition is free of polysaccharide based thickener in at least one embodiment.

Additionally or alternatively, the personal care composition may be essentially free of or free of thickener(s) (e.g., other than those constituting surfactants). For example, the personal care compositions may be formulated to have about 6 wt. % or less, about 4 wt. % or less, about 3 wt. % or less, about 2 wt. % or less, about 1 wt. % or less, about 0.5 wt. % or less, or about 0.1 wt. % or less, of thickener(s) (e.g., other than those constituting surfactants), based on the total weight of the personal care composition. The personal care composition is free of thickeners in at least one embodiment.

In certain embodiments, the personal care composition include salt in an amount ranging from about 0.1 to about 4.5 wt. %, based on the total weight of the personal care composition. For instance, the personal care composition may be formulated to have salt present in an amount from about 0.1 to about 4.5 wt. %, about 0.1 to about 4 wt. %, about 0.1 to about 3.5 wt. %, about 0.1 to about 3 wt. %, about 0.1 to about 2.5 wt. %, about 0.1 to about 2 wt. %; from about 0.5 to about 4.5 wt. %, about 0.5 to about 4 wt. %, about 0.5 to about 3.5 wt. %, about 0.5 to about 3 wt. %, about 0.5 to about 2.5 wt. %, about 0.5 to about 2 wt. %; from about 1 to about 4.5 wt. %, about 1 to about 4 wt. %, about 1 to about 3.5 wt. %, about 1 to about 3 wt. %, about 1 to about 2.5 wt. %, about 1 to about 2 wt. %; from about 1.5 to about 4.5 wt. %, about 1.5 to about 4 wt. %, about 1.5 to about 3.5 wt. %, about 1.5 to about 3 wt. %, about 1.5 to about 2.5 wt. %; from about 2 to about 4.5 wt. %, about 2 to about 4 wt. %, about 2 to about 3.5 wt. %, about 2 to about 3 wt. %; from about 2.5 to about 4.5 wt. %, about 2.5 to about 4 wt. %, about 2.5 to about 3.5 wt. %; from about 3 to about 4.5 wt. %, about 3 to about 4 wt. %, or any range or subrange thereof, based on the total weight of the personal care composition. The salt may be an inorganic salt, such as sodium chloride, sodium sulfate, calcium chloride, potassium chloride, or a combination thereof.

The personal care composition generally comprises water. Water may be present in the personal care composition in an amount ranging from about 45 to about 94 wt. %, based on the total weight of the personal care composition. For example, the personal care composition may comprise water in an amount from about 55 to about 94 wt. %, about 65 to about 94 wt. %, about 68 to about 94 wt. %, about 71 to about 94 wt. %, about 75 to about 94 wt. %, about 78 to about 94 wt. %, about 81 to about 94 wt. %, about 84 to about 94 wt. %; from about 45 to about 91 wt. %, about 55 to about 91 wt. %, about 65 to about 91 wt. %, about 68 to about 91 wt. %, about 71 to about 91 wt. %, about 75 to about 91 wt. %, about 78 to about 91 wt. %, about 81 to about 91 wt. %; from about 45 to about 88 wt. %, about 55 to about 88 wt. %, about 65 to about 88 wt. %, about 68 to about 88 wt. %, about 71 to about 88 wt. %, about 75 to about 88 wt. %, about 78 to about 88 wt. %; from about 45 to about 85 wt. %, about 55 to about 85 wt. %, about 65 to about 85 wt. %, about 68 to about 85 wt. %, about 71 to about 85 wt. %, about 75 to about 85 wt. %, about 78 to about 85 wt. %; from about 45 to about 82 wt. %, about 55 to about 82 wt. %, about 65 to about 82 wt. %, about 68 to about 82 wt. %, about 71 to about 82 wt. %, about 75 to about 82 wt. %; from about 45 to about 79 wt. %, about 55 to about 79 wt. %, about 65 to about 79 wt. %, about 68 to about 79 wt. %; from about 45 to about 76 wt. %, about 55 to about 76 wt. %, about 65 to about 76 wt. %, about 68 to about 76 wt. %, or any range or subrange thereof, based on the total weight of the personal care composition.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final form of personal care composition and/or product (e.g., in a non-diluted/concentrated form and/or in a diluted/ready-to-use form). For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

Without being limited to any particular theory, it is believed that personal care compositions formulated to have surfactant systems comprising certain surfactants in particular amounts and ratios disclosed herein with certain pHs can achieve preferable viscosities. The personal care compositions may have a pH from about 2 to about 4.5, about 2 to about 4, about 2 to about 3.75, about 2 to about 3.5, about 2 to about 3.25, about 2 to about 3; from about 2.5 to about 4.5, about 2.5 to about 4, about 2.5 to about 3.75, about 2.5 to about 3.5, about 2.5 to about 3.25, about 2.5 to about 3; from about 2.75 to about 4.5, about 2.75 to about 4, about 2.75 to about 3.75, about 2.75 to about 3.5, about 2.75 to about 3.25, or any range or subrange thereof.

The personal care compositions typically comprise a preservative system. The preservative system may comprise lactic acid, citric acid, sodium benzoate, salicylic acid, tetrasodium N,N-bis(carboxymethyl)-L-glutamate, a salt thereof, or a combination of two or more thereof. For example, the preservative system may include sodium lactate, sodium salicylate, calcium lactate, and/or sodium citrate. In some embodiments, the preservative system comprises citric acid and/or a salt thereof and salicylic and/or a salt thereof. In further embodiments, the preservative system comprises tetrasodium N,N-bis(carboxymethyl)-L-glutamate and at least one preservative selected from citric acid, salicylic, a salt thereof, and a combination of two or more thereof. Additionally or alternatively, the preservative system may include at least one of lactic acid, benzalkonium chloride, or a combination thereof, and at least one of sodium benzoate, potassium sorbate, hydroxyacetophenone, ethylhexylglycerin, or a combination of thereof. In some embodiments, the preservative system includes lactic acid, benzalkonium chloride, and at least one of sodium benzoate, potassium sorbate, and a combination of thereof.

The personal care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes. In some embodiments, the colorants may be present in an amount ranging from about 0.0001 to about 0.4 wt. %, including all percentages and subranges therebetween, based on the total weight of the personal care composition. In further embodiments, the colorants may be present in an amount ranging from about 0.0001 to about 4 wt. %, including all percentages and subranges therebetween, based on the total weight of the personal care composition.

### EXAMPLES

### Example 1

Two non-limiting, example personal care compositions (Example Compositions A and B) were prepared in accordance with aspects of the invention. Example Compositions A and B had a pH of 3.9 and 3.9-4.2, respectively, and were prepared according to the formulations shown in Table 1.

TABLE 1

| | Example | Example |
|---|---|---|
| | Composition A | Composition B |
| US INCI Compound Name | (wt. %) | (wt. %) |
| Sodium Methyl Cocoyl Taurate | 0.6 | 0.95 |
| Disodium 2-sulfolaurate | | 5.34 |
| Cocamidopropyl Betaine | 3.75 | 4 |
| Coco-Glucoside | 2.2 | 4.8 |
| Glyceryl Oleate | 0.5 | 0.5 |
| PEG-120 Methyl Glucose | | 0.2 |
| Dioleate | | |
| Glycerin | 2 | 2 |
| Sodium Lactate | 1.1 | |
| Lactic Acid | 0.643 | |
| Citric Acid | | 1 |
| Sodium Benzoate | | 0.28 |
| Sodium Salicylate | | 0.3 |
| Tetrasodium N,N- | | 0.21 |
| Bis(carboxymethyl)-L-glutamate | | |
| Fragrance | 0.2 | 0.72 |
| Water | Q.S. to 100 | Q.S. to 100 |

Example Composition A was evaluated pursuant to ISO Standard 16128 and determined to have a natural origin index of about 99.1%. Example Composition B was also evaluated pursuant to ISO Standard 16128 and determined to have a natural origin index of about 96.6%. Example Composition B was assessed and determined to have a biodegratability index of about 97%.

1. A personal care composition comprising:
from about 1 to about 26 wt. % of a surfactant system comprising:
a non-sulfate anionic surfactant,
an amphoteric surfactant, and
a nonionic surfactant; and
a polyol,
wherein the personal care composition has a pH of about 2 to about 4.5, and all weight percentages are based on the total weight of the personal care composition.

2. The personal care composition according to claim 1, wherein the personal care composition has a weight ratio of non-sulfate based anionic surfactant to amphoteric surfactant from about 1:2 to about 1:9.

3. The personal care composition according to claim 1, wherein the non-sulfate based anionic surfactant comprises sodium methyl cocoyl taurate, sodium methyl oleoyl taurate, or a combination thereof.

4. The personal care composition according to claim 1, wherein the amphoteric surfactant comprises cocamidopropyl betaine.

5. The personal care composition according to claim 1, wherein the nonionic surfactant comprises a glucoside surfactant, a glyceride surfactant, or a combination of two or more thereof.

6. The personal care composition according to claim 5, wherein the glyceride surfactant is selected from glycerol monostarate, glyceryl oleate, glyceryl linoleate, glyceryl ricinoleate, PEG glyceryl oleate, PCA glyceryl oleate, and a combination of two or more thereof.

7. The personal care composition according to claim 5, wherein the glucoside surfactant is selected from lauryl glucoside, lauroyl ethyl glucoside, myristoyl ethyl glucoside, oleoyl ethyl glucoside, and a combination of two or more thereof.

8. The personal care composition according to claim 5, wherein the nonionic surfactant comprises PEG methyl glucose dioleate.

9. The personal care composition according to claim 1, wherein the surfactant system is present in an amount from about 1 to about 20 wt. %, the non-sulfate anionic surfactant is present in an amount from about 0.3 to about 10 wt. %, the amphoteric surfactant is present in an amount from about 1 to about 9 wt. %, and the nonionic surfactant is present in an amount from about 1 to about 11 wt. %.

10. The personal care composition according to claim 1, wherein the polyol comprises ethylene glycol, propylene glycol, butylene glycol, hexylene glycol, glycerin, diglycerin, diethylene glycol, dipropylene glycol, a polyethylene glycol, a polypropylene glycol, a block polymer of polyethylene glycol and polypropylene glycol, or a combination of two or more thereof, or a combination of two more thereof.

11. The personal care composition according to claim 1 further comprising a preservative system comprising lactic acid, citric acid, sodium benzoate, sodium salicylate, tetrasodium N,N-bis(carboxymethyl)-L-glutamate, or a combination of two or more thereof.

12. The personal care composition according to claim 11, wherein the preservative system comprises lactic acid and/or a salt thereof.

13. The personal care composition according to claim 11, wherein the preservative system comprises tetrasodium N,N-bis(carboxymethyl)-L-glutamate and at least one preservative selected from citric acid, salicylic, a salt thereof, or a combination of two or more thereof.

14. The personal care composition according to claim 1, wherein the personal care composition is essentially free of or free of sulfate based anionic surfactants.

15. The personal care composition according to claim 1, wherein the personal care composition is essentially free of or free of ethylenediaminetetraacetic acid (EDTA).

16. The personal care composition according to claim 1, wherein the personal care composition has a natural origin index of about 90% or more pursuant to ISO Standard 16128.

17. The personal care composition according to claim 1, wherein the personal care composition has a biodegradability index of about 85% or more pursuant to ISO Standard 14855-1:2005.

18. A personal care composition comprising:
a surfactant system comprising PEG methyl glucose dioleate and one or more surfactant selected from:
a sulfonate surfactant,
a taurate surfactant,
a glyceride surfactant, or
a glycoside surfactant; and
a polyol.

19. A personal care composition comprising:
a surfactant system comprising:
a taurate surfactant,
a betaine surfactant, and
a glycoside surfactant;
a polyol,
wherein the personal care composition has a natural origin index of about 90% or more pursuant to ISO Standard 16128 and a biodegradability index of about 85% or more pursuant to ISO Standard 14855-1:2005.

20. The personal care composition according to claim 1, wherein the personal care composition has a viscosity of about 3,000 cP or more.
